(19) United States
(12) Patent Application Publication
(10) Pub. No.: US 2017/0228521 A1
(43) Pub. Date: Aug. 10, 2017

(54) REPORT DRIVEN WORKFLOW FOR OPHTHALMIC IMAGE DATA ACQUISITION

(71) Applicant: Carl Zeiss Meditec, Inc., Dublin, CA (US)

(72) Inventors: Raghavendra APPAKAYA, Bangalore (IN); Gautam JINDAL, Bangalore (IN); Arindam SARKER, Bangalore (IN); Ting ZHENG, Shanghai (CN); Xunchang CHEN, Shanghai (CN)

(21) Appl. No.: 15/016,719

(22) Filed: Feb. 5, 2016

(51) Int. Cl. G06F 19/00; A61B 3/14; A61B 3/00; G06K 9/00; A61B 3/10

(52) U.S. Cl. CPC G06F 19/3487; G06F 19/321; G06F 19/3431; G06K 9/00604; A61B 3/102; A61B 3/0025; A61B 3/14; A61B 3/0041

(57) ABSTRACT

An improved workflow for acquiring image data of the eye of a patient is described. The workflow can be used with any ophthalmic diagnostic devices including an Optical Coherence Tomography (OCT) device. This workflow is referred herein as a report driven workflow. Under the report driven workflow, a plurality of report options are presented to the device operator. These report options are selectable to generate a report summarizing analysis relating to a specific pathology or region of the eye. A desired report option is selected by the device operator. Based on the selected report option, one or more scan types are automatically selected by the device software. Image data corresponding to the one or more scan types are captured using the ophthalmic diagnostic device. An analysis for the selected desired report option is generated based on the captured image data and is then presented to the device operator.

### TECHNICAL FIELD

The present application relates to ophthalmic image data acquisition of a patient using an improved workflow, in particular systems and methods for acquiring and displaying a series of ophthalmic image data of the eye of the patient using a report driven workflow.

### BACKGROUND

In commercially available ophthalmic diagnostic systems, an instrument operator typically selects from a series of scanning options based on known locations in the eye that may be relevant to a specific pathology. While performing scans using these ophthalmic diagnostic systems, in particular using an optical coherence tomography (OCT) system, the instrument operator spends a lot of time in selecting an appropriate scan type and then creating the reports that summarize the results. This process or workflow is time consuming and sometimes results in a novice operator selecting a wrong scan type. US Patent Publication No. 2014/0293222, the contents of which are hereby incorporated by reference, discloses a workflow (referred as a protocol driven workflow) including a user interface where the instrument operator has the ability to order single exams, or a particular protocol which is a combination of one or more scans, or analysis from a single or multiple diagnostic devices. This protocol driven workflow could be based on desired information on a specific disease state such as glaucoma or dry or wet AMD. This workflow also implies that the user can order the protocol for a particular patient at the end of the examination for their next visit. The information will be stored and recalled the next time the patient is examined. Here we describe further improvements to ophthalmic diagnostic instrument workflows that could be implemented in any ophthalmic diagnostic systems and that makes the patient scanning process fast, easy to use, and reduces or eliminates errors related to wrong scan selection.

### SUMMARY

It is an object of the present invention to improve the user workflow in OCT and other ophthalmic diagnostic devices. With the use of such an improved workflow, the device operator does not need to worry about selecting particular scan types and just needs to select a report option for the report that he/she desires. The desired report will include results of an analysis or diagnosis relating to a specific pathology (e.g., Glaucoma, AMD, etc.) or region (e.g., anterior segment, posterior segment, etc.) of the eye of a patient. Based on the selection of the report option, the device software will automatically determine and guide the acquisition of the appropriate scan types. In some instances, the improved workflow described herein is also referred to as a report driven workflow.

The report driven workflow is advantageous in a number of respects. For instance, it reduces or eliminates the errors that arise from operator's wrong selection of scan types. Further, the operator does not need to remember which scan type should be selected for a desired report. Yet further, the report driven workflow can be used for any diagnostic devices, which have multiple scan types and reports corresponding to a selected scan type. It should be understood that the foregoing advantages are provided by way of example and other advantages and/or benefits are also possible and contemplated.

### BRIEF DESCRIPTION OF THE FIGURES

FIG. 1 shows a generalized diagram of an ophthalmic OCT device that can be used in various embodiments of the present invention.

FIG. 2 is a flowchart of an example report driven workflow that can be used in OCT and other ophthalmic diagnostic devices according to one aspect of the present invention.

FIG. 3a is a graphical user interface (GUI) for selecting one or more desired report options for image data acquisition according to one aspect of the present invention.

FIG. 3b is a GUI for viewing, capturing, and optimizing image data according to one aspect of the present invention.

FIG. 3c is a GUI for viewing a generated analysis based on captured image data and printing results of the analysis as one or more reports according to one aspect of the present invention.

FIG. 3d shows an example analysis report according to one aspect of the present invention.

### DETAILED DESCRIPTION

Optical Coherence Tomography (OCT) is a technique for performing high-resolution cross-sectional imaging that can provide images of tissue structure on the micron scale in situ and in real time. OCT is a method of interferometry that determines the scattering profile of a sample along the OCT beam. Each scattering profile is called an axial scan, or A-scan. Cross-sectional images (B-scans), and by extension 3D volumes, are built up from many A-scans, with the OCT beam moved to a set of transverse locations on the sample.

In time-domain OCT (TD-OCT), an optical delay line is used for mechanical depth scanning with a relatively slow imaging speed. In frequency domain OCT (FD-OCT), the interferometric signal between light from a reference and the back-scattered light from a sample point is recorded in the frequency domain rather than the time domain. After a wavelength calibration, a one-dimensional Fourier transform is taken to obtain an A-line spatial distribution of the object scattering potential. The spectral information discrimination in FD-OCT is typically accomplished by using a dispersive spectrometer in the detection arm in the case of spectral-domain OCT (SD-OCT) or rapidly scanning a swept laser source in the case of swept-source OCT (SS-OCT).

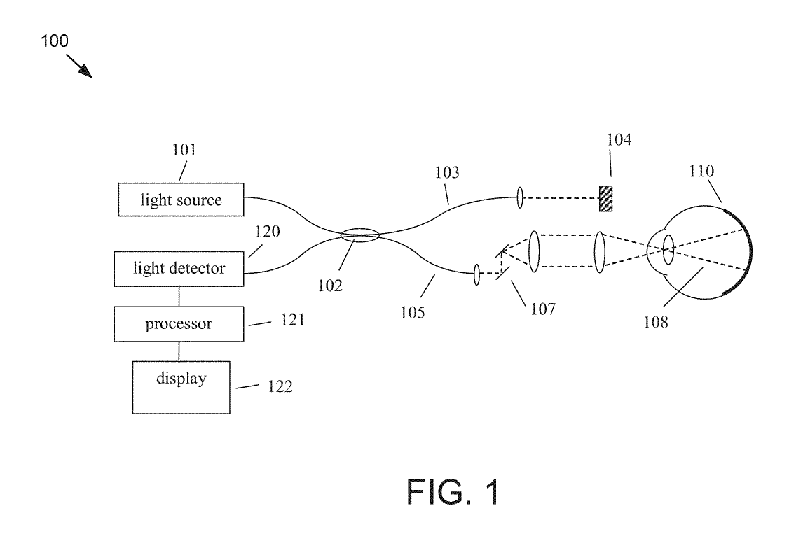

Evaluation of biological materials using OCT was first disclosed in the early 1990's. Frequency domain OCT techniques have been applied to living samples. The frequency domain techniques have significant advantages in speed and signal-to-noise ratio as compared to time domain OCT. The greater speed of modern OCT systems allows the acquisition of larger data sets, including 3D volume images of human tissue. The technology has found widespread use in ophthalmology. A generalized FD-OCT system used to collect 3-D image data of the eye suitable for use with the present invention is illustrated in FIG. 1.

A FD-OCT system 100 includes a light source, 101, typical sources including but not limited to broadband light sources with short temporal coherence lengths or swept laser sources. A beam of light from source 101 is routed, typically by optical fiber 105, to illuminate the sample 110, a typical sample being tissues in the human eye. The source 101 can be either a broadband light source with short temporal coherence length in the case of SD-OCT or a wavelength tunable laser source in the case of SS-OCT. The light is scanned, typically with a scanner 107 between the output of the fiber and the sample, so that the beam of light (dashed line 108) is scanned laterally (in x and y) over the region of the sample to be imaged. Light scattered from the sample is collected, typically into the same fiber 105 used to route the light for illumination. Reference light derived from the same source 101 travels a separate path, in this case involving fiber 103 and retro-reflector 104 with an adjustable optical delay. Those skilled in the art recognize that a transmissive reference path can also be used and that the adjustable delay could be placed in the sample or reference arm of the interferometer. Collected sample light is combined with reference light, typically in a fiber coupler 102, to form light interference in a detector 120. Although a single fiber port is shown going to the detector, those skilled in the art recognize that various designs of interferometers can be used for balanced or unbalanced detection of the interference signal. The output from the detector 120 is supplied to a processor 121 that converts the observed interference into depth information of the sample. The results can be stored in the processor 121 or other storage medium or displayed on display 122. The processing and storing functions may be localized within the OCT instrument or functions may be performed on an external processing unit to which the collected data is transferred. This unit could be dedicated to data processing or perform other tasks which are quite general and not dedicated to the OCT device. The processor 121 may contain for example a field-programmable gate array (FPGA), a digital signal processor (DSP), an application specific integrated circuit (ASIC), a graphics processing unit (GPU), a system on chip (SoC) or a combination thereof, that performs some, or the entire data processing steps, prior to passing on to the host processor or in a parallelized fashion.

The interference causes the intensity of the interfered light to vary across the spectrum. The Fourier transform of the interference light reveals the profile of scattering intensities at different path lengths, and therefore scattering as a function of depth (z-direction) in the sample. The profile of scattering as a function of depth is called an axial scan (A-scan). A set of A-scans measured at neighboring locations in the sample produces a cross-sectional image (tomogram or B-scan) of the sample. A collection of B-scans collected at different transverse locations on the sample makes up a data volume or cube. For a particular volume of data, the term fast axis refers to the scan direction along a single B-scan whereas slow axis refers to the axis along which multiple B-scans are collected. A variety of ways to create B-scans are known to those skilled in the art including but not limited to along the horizontal or x-direction, along the vertical or y-direction, along the diagonal of x and y, or in a circular or spiral pattern.

The sample and reference arms in the interferometer could consist of bulk-optics, fiber-optics or hybrid bulk-optic systems and could have different architectures such as Michelson, Mach-Zehnder or common-path based designs as would be known by those skilled in the art. Light beam as used herein should be interpreted as any carefully directed light path. In time-domain systems, the reference arm needs to have a tunable optical delay to generate interference. Balanced detection systems are typically used in TD-OCT and SS-OCT systems, while spectrometers are used at the detection port for SD-OCT systems. The invention described herein could be applied to any type of OCT system including OCT systems that collect scans in parallel configurations including line-field, partial-field and full-field. Various aspects of the invention could apply to other types of ophthalmic diagnostic systems and/or multiple ophthalmic diagnostic systems including but not limited to fundus imaging systems, visual field test devices, and scanning laser polarimeters. The invention relates to acquisition controls, processing and display of ophthalmic image (or other diagnostic) data that can be done on a particular instrument itself or on a separate computer or workstation to which collected image (or other diagnostic) data is transferred either manually or over a networked connection. The display provides a graphical user interface for the instrument or operator to interact with the system and resulting data. Some aspects of OCT user interfaces are described in US Patent Publication No. 2008/0100612, the contents of which are hereby incorporated by reference. The instrument user can interact with the interface and provide input in a variety of ways including but not limited to, mouse clicks, touchscreen elements, scroll wheels, buttons, knobs, etc. The invention described herein is directed towards improvements in how the user interface is designed and configured to allow for optimized acquisition, display and analysis of ophthalmic image data.

Report Driven Workflow

Typically for OCT imaging, a specific scan type or series of scans will be selected by the instrument user and performed on the patient. Existing workflow requires knowledge of what scan types are likely to provide the desired information for analysis. Once the scan types are selected, the instrument user can proceed to acquire image data (e.g., B-scans, fundus images, etc.), analyze information acquired in the image data, and create the reports summarizing the analysis. This way of scan acquisition is usually time consuming. Furthermore, a novice instrument user is often unfamiliar with the different scan types and can end up selecting a wrong scan type. An aspect of the present invention is to improve or simplify the existing workflow which enables the instrument user to automatically obtain scan types and corresponding image data by just selecting report options for the reports that he/she requires. Such a simplified or improved workflow, as mentioned elsewhere herein, is referred to as a report driven workflow.

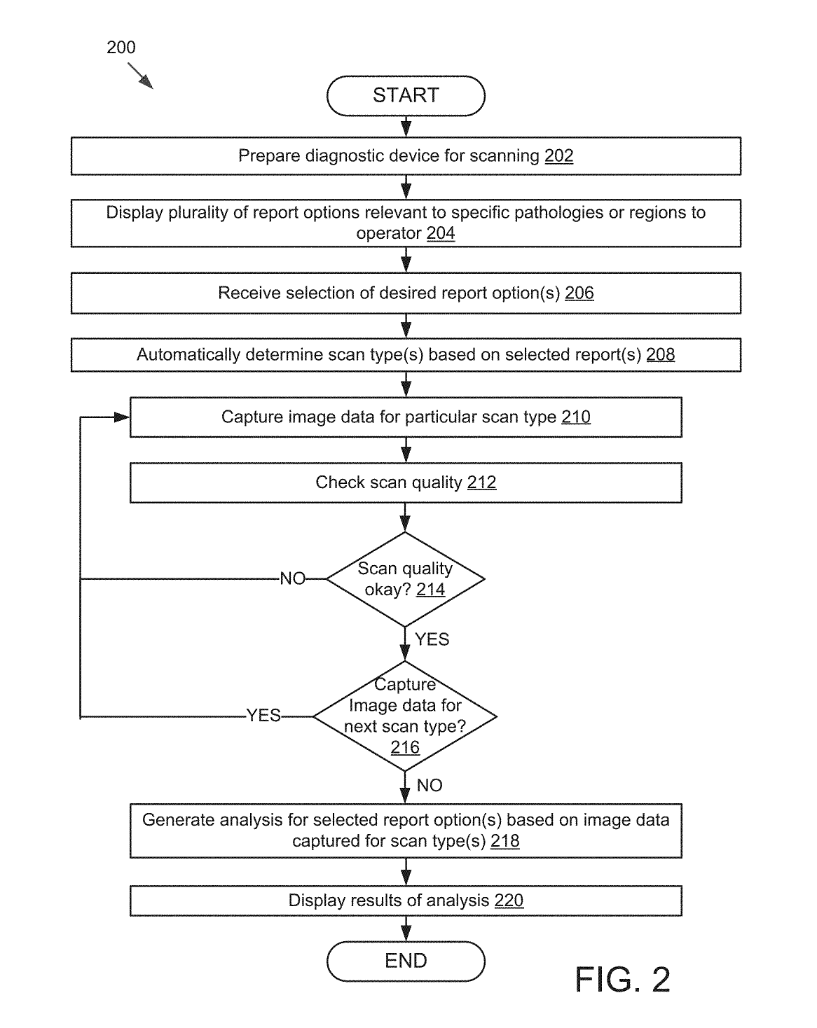

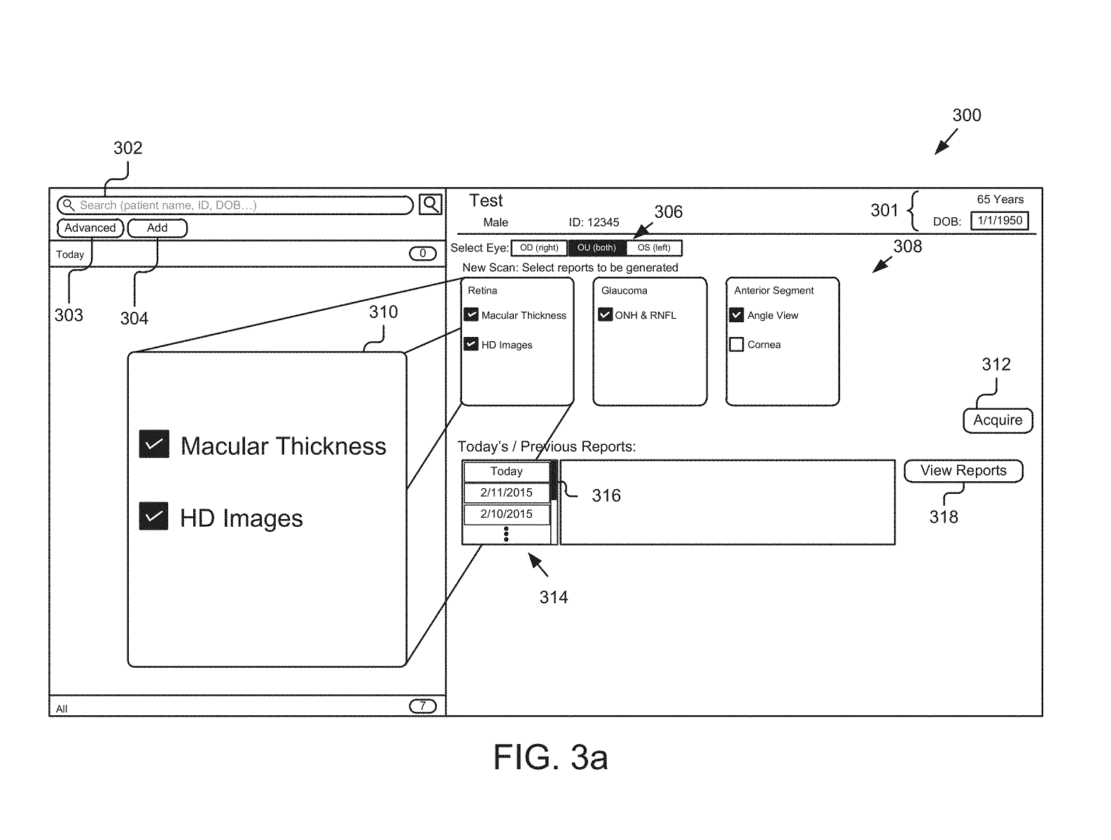

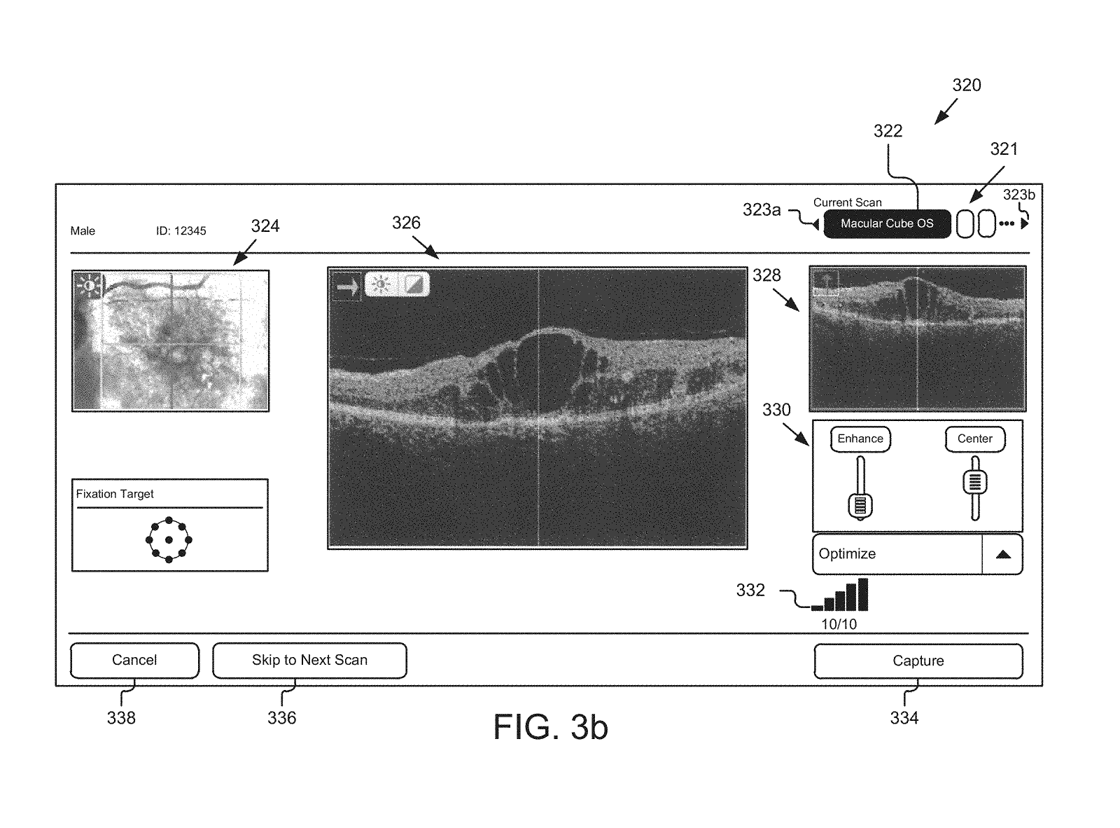

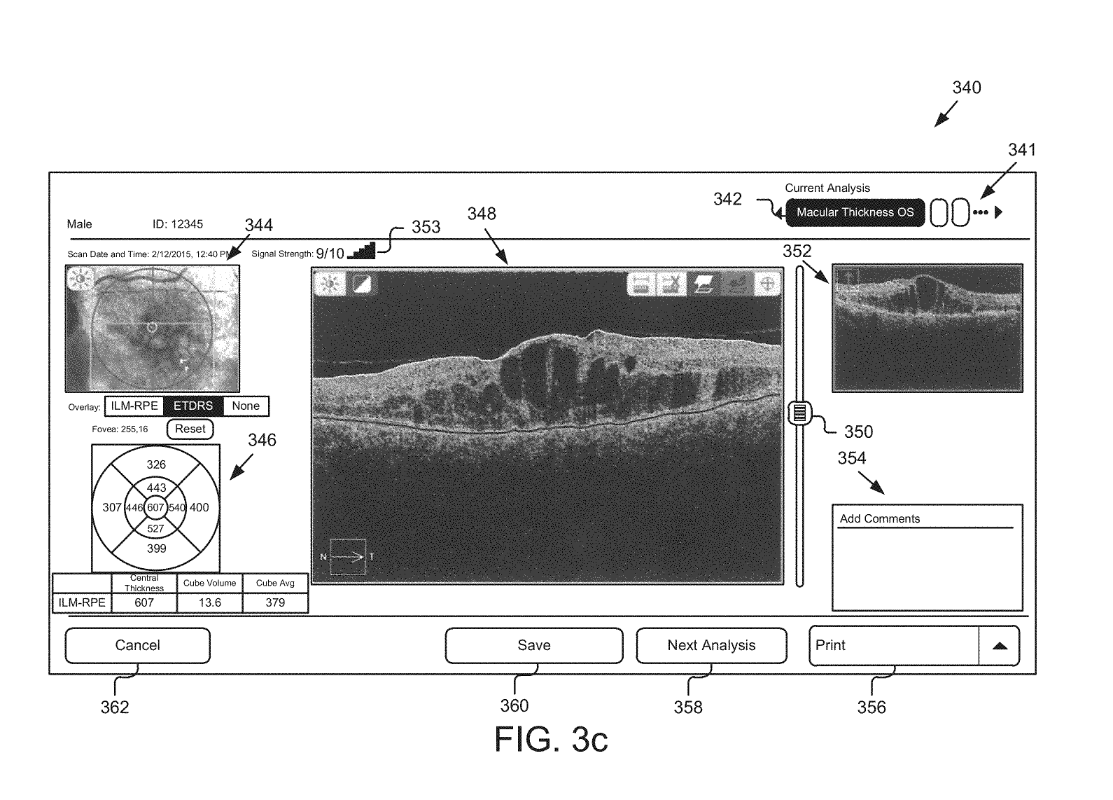

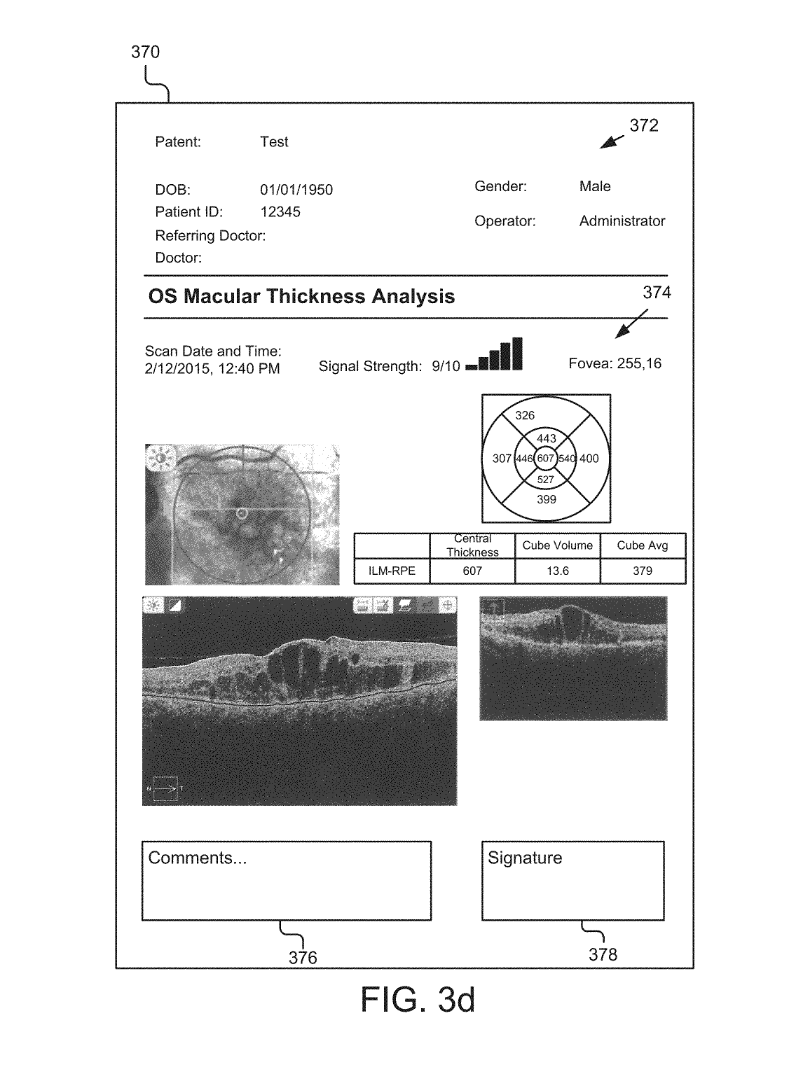

FIG. 2 is a flowchart that depicts steps involved in an example of a report driven workflow 200. Step 202 involves preparing an ophthalmic diagnostic device (e.g., OCT device) for scanning. Preparing the ophthalmic diagnostic device for scanning may include powering on the device, entering device operator's login details (e.g., username and password), retrieving patient details from a database using an existing patient ID, and/or creating a new patient ID. Once the device is fully set up for scanning, in step 204, the device will display a plurality of report options to the device operator. Each of these report options is selectable for generating a report at the end of the workflow that a user (e.g., device operator, patient) desires. The desired report will include a summary of an analysis or diagnosis relating to a specific disease (e.g., Glaucoma, dry or wet AMD, etc.), sample (e.g., retina, cornea, etc.) and/or a region or portion (e.g., anterior segment, posterior segment, etc.) of the eye of the patient. For example, the report may be a Macular Thickness report, a ONH & RFNL report, a high definition (HD) images report including analysis relating to a 5 Line Raster or a 1 Line Raster, etc. In step 206, the device may receive operator's selection of one or more report options, either for a single eye or for both the eyes of the patient, for the one or more reports that the operator wants to generate. By way of example and with reference to FIG. 3a, different report options for Glaucoma, Retina, and Anterior segment may be offered to the operator and the operator can select one or more reports from these report options that he/she desires and that are relevant for patient's examination. The operator does not need to remember or know different scan types and just needs to select the desired report options. The report option selection can be achieved by a mouse click or a touch screen or any other type of user input device well known to someone skilled in the art.

In step 208, the ophthalmic device software, such as OCT software, will automatically determine one or more appropriate scan types based on the one or more selected report options in step 206 (see, for example, reference numeral 321 in FIG. 3b). Some of the exemplary scan types that may be determined based on report options selection include, without limiting, Macular Cube 512×32, Macular Cube 512×128, Macular Cube 200×200, 5 Line Raster, 1 Line Raster, Optic Disc Cube 128×128, Optic Disc Cube 200×200, etc. By way of example, upon selecting the macular thickness report option in FIG. 3a for left eye (OS), the device software will automatically determine its corresponding scan type “Macular Cube OS” as depicted in FIG. 3b. As another example, upon selecting the “HD Images” option in FIG. 3a, the device software may automatically determine the scan type as a 5 line raster or a 1 line raster. This is advantageous from the previous workflow design because in the previous workflow a user was required to first select a scan type, which in this case the “Macular Cube OS” and then select “Macular Thickness” option for analysis and generating the report. Furthermore, in the previous workflow, the user performed scans and analysis of the scans for different pathologies or regions of the eye one by one. Whereas with the improved workflow discussed herein, the user can choose to scan, analyze, and generate multiple reports for different pathologies or regions of the eye all at one time. Continuing with the method 200, the operator may instruct the patient to blink the eye before capturing the image data. In step 210, for a particular scan type, the operator can capture the image data (e.g., B-scans) by pressing on the joy stick or mouse click. For example, as shown in FIG. 3b, the operator can capture a fundus image 324, a large B-scan 326, and a small vertical B-scan 328 for the scan type “Macular Cube OS” 322 corresponding to the left eye of the patient by clicking on the capture button 334. In some instances, the device will beep when the scan acquisition is completed. Alternatively, the device could acquire the image data automatically without user intervention. In step 212, the operator can see the acquired image data (e.g., either as the data is acquired for the current scan type or all at once for all the selected scan types) and decide on scan quality (step 214). The device can display real time signal strength of the captured scan. The signal strength may be a value between a range of 1 to 10 to indicate the quality of the captured scan, where 1 being the worst quality and 10 being the best quality. In some embodiments, there may be a threshold value in the range that may be used to indicate if image data need to be recaptured. For instance, the threshold value may be 6. If the signal strength is less than the threshold value, the device will advise the operator to rescan the patient and the operator can then re-acquire image data for the same scan type by repeating the step 210. If on the other hand, the signal strength is greater than the threshold value and the operator confirms that he/she is satisfied with the scan quality, then the OCT workflow software will guide the operator to next step 216 to capture image data for the next scan type (step 210). The next scan type may correspond to the next report in the workflow. For instance, if two or more reports were selected in step 206, then the OCT software will guide the operator to acquire image data corresponding to the next report in the workflow. In some embodiments, a same scan type may be used to acquire image data for multiple reports in the workflow. By way of an example, the scan type determined for acquiring image data corresponding to 5 Line HD Raster may also be used for acquiring image data corresponding to 1 Line HD. The system can display an indication of the scan or image data acquisition progress to the user, as shown for example by reference numeral 321 in FIG. 3b.

In some embodiments, the software may instruct the user to install or remove auxiliary optics, typically one or more lenses, to the system to enable different imaging modes as part of the workflow (see for example US Patent Publication No. 2014/0268039 and US Patent Publication No. 2015/0085294, the contents of both of which are hereby incorporated by reference). OCT systems commonly require a lens assembly to be added to the exterior of the system in addition to an adjustment to the delay between reference and sample arms to switch between imaging structures in the anterior and posterior of the eye. Auxiliary lenses can also be used to change the field of view within a particular region of the eye.

Once all the image data for the selected report options are acquired, the OCT workflow software, in step 218, will generate an analysis and in step 220, display the analysis to the device operator, for example, the analysis screen as shown in FIG. 3c. The analysis screen may enable the operator to view and measure anatomical structures depicted in the acquired image data for each report option in the one or more report options selected by the operator. The operator can navigate to analyze the acquired image data for each report option in the workflow by a mouse click or any other input means. The device can display an indication of the analysis progress to the user, as shown for example by reference numeral 342 in FIG. 3c. The operator can choose to generate and/or print one or more reports summarizing one or more results of the analysis. For instance, the operator can choose to generate a macular thickness report (see FIG. 3d) that may summarize the results of the macular thickness analysis, as shown for example in FIG. 3c. The one or more reports can further be exported in PDF file format to a USB or other connected devices or the patient's electronic health record. By way of illustration, FIG. 3d shows an example macular thickness analysis report 370. The report 370 includes patient information 372 such as patient's name, date of birth, patient ID, gender, etc. The report 370 further includes results of an analysis (indicated by reference numeral 374) and one or more comments (as indicated by reference numeral 376) that were entered by the operator during the analysis, such as the macular thickness analysis as shown in FIG. 3c. The device operator can choose to print a hardcopy of the report 370 and then authorize it by signing in the signature box 380. As mentioned elsewhere herein, the operator can export the report in PDF format to a USB, save the report in hard drive for future access and/or retrieval, or can further burn it to a CD/DVD.

FIG. 3a is a graphical user interface (GUI) 300 for displaying and selecting one or more desired report options for image data acquisition according to one aspect of the present invention. The GUI 300 is shown once the diagnostic device associated with it is prepared with all the initial steps, which includes powering on the device, entering the login details of the operating user, and entering patient information such as age and date of birth, as shown by reference numeral 301. The operating user can search an existing patient by entering patient name, ID, or date of birth in the search box 302 or can add a new patient for scanning using the add button 304. Although not shown in the figure, upon activating the add button 304, the operating user can enter the new patient's first name, last name, gender, age, and date of birth in order to add the patient for scanning. On clicking the advanced button 303, the operating user will be able to view and select more options.

The operator can select which eye he/she wants to scan of the patient. As indicated by reference numeral 306, the operator can choose to scan either the left eye, the right eye, or both the eyes of the patient. Scan report options for Glaucoma, Retina, and Anterior segment are offered to the operator, as shown by reference numeral 308. Each of these three areas includes different report options that the operator can select to generate the reports. For instance and as depicted zoomed in by 310, the operator selects the “Macular Thickness” and the “HD Images” report options for scanning the Retina and generating a corresponding macular thickness report and a HD images report. Once the operator is done selecting all the desired report options, the operator can go ahead and start the acquisition of the corresponding image data by selecting the acquire button 312. In one embodiment, the report options may be processed one at a time in the left to right and top to bottom order. So in this particular case, the selected report options for Retina (Macular Thickness→HD Images→ . . . ) will be processed first, then the report option for Glaucoma, and finally the report option for Anterior Segment. The GUI 300 also gives the operator the ability to view reports that were generated at the current or any previous visits of the patient, as shown by reference numeral 314. The operator can scroll the list using 316 to select one or more reports and then view them using the button 318. In some instances, the operator can select a previous report and the report options in the report selection region 308 will be automatically selected for the current image data acquisition of a patient based on the reports that were generated in the last patient visit. This is advantageous for a doctor who wants to perform the same scans for a particular region of his/her patient's eye in order to observe any changes from the last patient visit.

FIG. 3b is a GUI 320 for capturing and optimizing image data according to one aspect of the present invention. The image data correspond to the images captured in real-time using an ophthalmic diagnostic device (e.g., OCT device) for a particular scan type. The GUI 320 includes a status bar 321 that indicates the current scan type 322 in the acquisition process and the number of scan types that are left to process based on the number of report options selected by the operator, for example the report options selected in FIG. 3a. The operator can view the previously processed scan types and the upcoming scan types using the scroll buttons 323a and 323b, respectively. In the particular depicted scenario, the current scan type 322 in the acquisition process is the “Macular Cube OS” and the GUI 320 shows the image data including a fundus image 324, a large horizontal B-scan 326, and a vertical B-scan 328 corresponding to that scan type. The locations of the displayed B-scans are indicated by the horizontal and vertical lines on the fundus image and each B-scan contains a horizontal line indicating the location of the other displayed B-scan. For cube scans containing multiple B-scans, additional B-scans from the cube can be displayed by moving any of the lines displayed on the three images. As mentioned elsewhere herein, the scan type may be automatically selected based on a report option selected by the device operator, for example based on the “Macular Thickness” report option selected in FIG. 3a. The operator can optimize (e.g., enhance or centralize) the image data using adjustable scroll bars depicted by reference numeral 330. Reference numeral 332 indicates a signal strength value that is associated with the image data. In this particular case, the signal strength is excellent as indicated by its value 10/10. In some instances, if the signal strength is lower than value 6, then the device will automatically advise the operator to re-capture the scans. Once the operator is satisfied with the image data quality, he/she may capture the image data by clicking on the capture button 334. If instead, the operator wants to skip the current scan type and wants to capture image data for a next scan type, then he/she may do by clicking on the “Skip to Next Scan” button 336. If at any point in time, the operator wishes to cancel the image data acquisition process and return to the main screen, he/she may do so by clicking on the “Cancel” button 338.

FIG. 3c is a GUI 340 for viewing a generated analysis based on captured image data and printing results of the analysis as one or more reports according to one aspect of the present invention. The GUI 340 depicts results of the analysis generated for the report option “Macular Thickness OS”, as indicated by reference numeral 342. The results of the generated analysis include a fundus image 344 with options to overlay inner limiting membrane—retinal pigment epithelium (ILM-RPE) thickness map or move the Early Treatment of Diabetic Retinopathy Study (ETDRS) macular map sectors to a desired foveal location, an ETDRS measurement grid 346 to automatically and accurately locate the fovea, a horizontal B-scan viewer 348 with an associated scroll bar 350 for switching between different horizontal B-scans in the viewer 348, and a vertical B-scan 352. The locations of the displayed B-scans can be indicated by lines on the fundus image. The GUI 340 further includes a status bar 341 that indicates the current scan analysis 342 for a particular report option and the number of other generated analysis in the queue based on the number of report options selected by the operator, for example the report options selected in FIG. 3a. Reference numeral 353 indicates a signal strength value that is associated with these scans. In this particular case, the signal strength is very good as indicated by its value 9/10. The operator can enter his/her comments to include in the patient's report or choose from predefined comments using the dialog box 354. Once the operator is done entering the comments and analyzing the captured image data, he/she may print the results of the analysis as a report by clicking on the print button 356 and then choosing to print as a report. FIG. 3d shows an example macular thickness analysis report 370 for the left eye (OS) that includes the results of the analysis, as indicated by reference numeral 374. The operator can choose to print the report in the PDF format or any other types. In some instances, the operator can also export the report to a USB or any other connected devices or the patient's electronic health record. If the operator wants to skip the current analysis and wants to move to analysis of next report option, then he/she may do by clicking on the “Next Analysis” button 358. The operator can also save the current analysis for later viewing by clicking on the “Save” button 360. If at any point in time, the operator wishes to cancel the analysis process and return to the main screen, he/she may do so by clicking on the “Cancel” button 362.

Although various applications and embodiments that incorporate the teachings of the present invention have been shown and described in detail herein, those skilled in the art can readily devise other varied embodiments that still incorporate these teachings.

What is claimed is:

1. A method of acquiring image data of the eye of a patient with an ophthalmic diagnostic device, said method comprising:
displaying a plurality of report options to a user operating the ophthalmic diagnostic device, each report option being selectable to generate a report summarizing analysis relating to a specific pathology or region of the eye;
receiving a selection of a desired report option from the user;
automatically selecting one or more appropriate scan types based on the selected desired report option; and
capturing the image data of the eye of the patient using the ophthalmic diagnostic device, the image data corresponding to the one or more appropriate scan types;
generating analysis for the selected desired report option based on the captured image data; and
displaying or storing results of the analysis or a further processing thereof.

2. A method as recited in claim 1, said method further comprising:
generating a report based on the results of the analysis.

3. A method as recited in claim 1, said method further comprising:
checking the quality of the image data captured for each scan type based on a signal strength value.

4. A method as recited in claim 3, wherein checking the quality of the image data based on the signal strength value comprises:
approving the image data if the signal strength value is greater than a certain threshold value; and
advising the operator to re-capture the image data if the signal strength is lower than the certain threshold value.

5. A method as recited in claim 1, said method further comprising:
notifying the user to install or remove one or more auxiliary optics in the ophthalmic diagnostic device to switch between different imaging modes for capturing image data based on the selected desired report option.

6. A method as recited in claim 1, wherein the report is a macular thickness report, a high definition (HD) images report, an optic nerve hypoplasia (ONH) and retinal nerve fiber layer (RNFL) report, an angle view report, and a cornea report.

7. A method as recited in claim 1, wherein the one or more scan types are macular cube 512×32, macular cube 512×128, macular cube 200×200, 5 line raster, 1 line raster, optic disc cube 128×128, and optic disc cube 200×200.

8. A method as recited in claim 6, wherein the HD images report includes image data corresponding to a 5 line raster or a 1 line raster scan type.

9. A method as recited in claim 1, wherein the image data include one or more of a fundus image, a horizontal B-scan, and a vertical B-scan.

10. A method as recited in claim 1, wherein the ophthalmic diagnostic device is an optical coherence tomography (OCT) device.

11. A method of acquiring image data of the eye of a patient with an ophthalmic diagnostic device, said method comprising:
displaying a plurality of report options to a user operating the ophthalmic diagnostic device, each report option being selectable to generate a report summarizing analysis relating to a specific pathology or region of the eye;
receiving a selection of two or more report options from the user;
automatically selecting one or more scan types for each selected report option; and
capturing the image data corresponding to each scan type of the eye of the patient using the ophthalmic diagnostic device;
generating analysis for the two or more reports options based on the captured image data; and
displaying or storing results of the analysis for each selected report option or a further analysis thereof.

12. A method as recited in claim 11, said method further comprising:
generating one or more reports based on the results of the analysis.

13. A method as recited in claim 11, wherein the one or more scan types for the two or more report options are same.

14. A method as recited in claim 11, said method further comprising:
notifying the user to install or remove one or more auxiliary optics in the ophthalmic diagnostic device to switch between different imaging modes for capturing image data based on each of the selected two or more report options.

15. A method as recited in claim 11, said method further comprising:
checking the quality of the image data captured for each scan type based on a signal strength value.

16. A method as recited in claim 15, wherein checking the quality of the image data based on the signal strength value comprises:
approving the image data if the signal strength value is greater than a certain threshold value; and
advising the operator to re-capture the image data if the signal strength is lower than the certain threshold value.

17. A method as recited in claim 11, wherein the ophthalmic diagnostic device is an optical coherence tomography (OCT) device.

18. An ophthalmic diagnostic device for acquiring image data of the eye of a patient, said device comprising:
a light source for generating a beam of light;
optics for illuminating the eye with the beam of light;
a detector for measuring light returning from the eye and generating signals in response thereto;
a display with a graphical user interface for displaying a plurality of report options to a user operating the ophthalmic diagnostic device, each report option being selectable to generate a report summarizing analysis relating to a specific pathology or region of the eye;
an input device for receiving a selection of one or more desired report options from the user; and
a processor for automatically selecting one or more appropriate scan types based on the one or more desired report options, said processor further configured for generating image data based on the signals from the detector and for generating an analysis for the one or more desired report options based on the image data, and wherein said display is further configured for displaying results of the analysis to the user.

19. An ophthalmic diagnostic device as recited in claim 18, wherein:
said processor is further configured for generating one or more reports based on the results of the analysis.

20. An ophthalmic diagnostic device as recited in claim 19, said device further comprising:
a memory for storing the one or more reports for future access or retrieval.

21. An ophthalmic diagnostic device as recited in claim 18, said device further comprising:
an auxiliary lens for switching between different imaging modes for capturing the image data based on the selected one or more report options.

22. An ophthalmic diagnostic device as recited in claim 18, wherein said device is an optical coherence tomography (OCT) device.
